SEQUENCE LISTING (11 sequences)

SEQ ID NO: 1 - DNA, 1484 bp, Human
cccgtgaggaggaaaaggtgtgtccgctgccacccagtgtgagcgggtgacaccacccggttaggaaatcccagctcccaagagggtataaatccctgctttactgctgagctcctgctggaggtgaaagtctggcctggcagccttccccaggtgagcagcaacaaggccacgtgctgctgggtctcagtcctccacttcccgtgtcctctggaagttgtcaggagcaatgttgcgcttgtacgtgttggtaatgggagtttctgccttcacccttcagcctgcggcacacacaggggctgccagaagctgccggtttcgtgggaggcattacaagcgggagttcaggctggaaggggagcctgtagccctgaggtgcccccaggtgccctactggttgtgggcctctgtcagcccccgcatcaacctgacatggcataaaaatgactctgctaggacggtcccaggagaagaagagacacggatgtgggcccaggacggtgctctgtggcttctgccagccttgcaggaggactctggcacctacgtctgcactactagaaatgcttcttactgtgacaaaatgtccattgagctcagagtttttgagaatacagatgctttcctgccgttcatctcatacccgcaaattttaaccttgtcaacctctggggtattagtatgccctgacctgagtgaattcacccgtgacaaaactgacgtgaagattcaatggtacaaggattctcttcttttggataaagacaatgagaaatttctaagtgtgagggggaccactcacttactcgtacacgatgtggccctggaagatgctggctattaccgctgtgtcctgacatttgcccatgaaggccagcaatacaacatcactaggagtattgagctacgcatcaagaaaaaaaaagaagagaccattcctgtgatcatttcccccctcaagaccatatcagcttctctggggtcaagactgacaatcccgtgtaaggtgtttctgggaaccggcacacccttaaccaccatgctgtggtggacggccaatgacacccacatagagagcgcctacccgggaggccgcgtgaccgaggggccacgccaggaatattcagaaaataatgagaactacattgaagtgccattgatttttgatcctgtcacaagagaggatttgcacatggattttaaatgtgttgtccataataccctgagttttcagacactacgcaccacagtcaaggaagcctcctccacgttctcctggggcattgtgctggccccactttcactggccttcttggttttggggggaatatggatgcacagacggtgcaaacacagaactggaaaagcagatggtctgactgtgctatggcctcatcatcaagactttcaatcctatcccaagtgaaataaatggaatgaaataattcaaacacaaaaaaaaaaaaaaaaaaaaaaaaaaaaaa

SEQ ID NO: 2 - DNA, 1430 bp, Human
gggatgggagatactgttgtggtcacctctggaaaatacattctgctactcttaaaaactagtgacgctcatacaaatcaacagaaagagcttctgaaggaagactttaaagctgcttctgccacgtgctgctgggtctcagtcctccacttcccgtgtcctctggaagttgtcaggagcaatgttgcgcttgtacgtgttggtaatgggagtttctgccttcacccttcagcctgcggcacacacaggggctgccagaagctgccggtttcgtgggaggcattacaagcgggagttcaggctggaaggggagcctgtagccctgaggtgcccccaggtgccctactggttgtgggcctctgtcagcccccgcatcaacctgacatggcataaaaatgactctgctaggacggtcccaggagaagaagagacacggatgtgggcccaggacggtgctctgtggcttctgccagccttgcaggaggactctggcacctacgtctgcactactagaaatgcttcttactgtgacaaaatgtccattgagctcagagtttttgagaatacagatgctttcctgccgttcatctcatacccgcaaattttaaccttgtcaacctctggggtattagtatgccctgacctgagtgaattcacccgtgacaaaactgacgtgaagattcaatggtacaaggattctcttcttttggataaagacaatgagaaatttctaagtgtgagggggaccactcacttactcgtacacgatgtggccctggaagatgctggctattaccgctgtgtcctgacatttgcccatgaaggccagcaatacaacatcactaggagtattgagctacgcatcaagaaaaaaaaaagaagagaccattcctgtgatcatttcccccctcaagaccatatcagcttctctggggtcaagactgacaatcccgtgtaaggtgtttctgggaaccggcacacccttaaccaccatgctgtggtggacggccaatgacacccacatagagagcgcctacccgggaggccgcgtgaccgaggggccacgccaggaatattcagaaaataatgagaactacattgaagtgccattgatttttgatcctgtcacaagagaggatttgcacatggattttaaatgtgttgtccataataccctgagttttcagacactacgcaccacagtcaaggaagcctcctccacgttctcctggggcattgtgctggccccactttcactggccttcttggttttggggggaatatggatgcacagacggtgcaaacacagaactggaaaagcagatggtctgactgtgctatggcctcatcatcaagactttcaatcctatcccaagtgaaataaatggaatgaaataattcaaaaaaaaaaaaaaaaaaaaaaaaaaaaa

SEQ ID NO: 3 - protein, 398 aa, Human
MLRLYVLVMGVSAFTLQPAAHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKEASSTFSWGIVLAPLSLAFLVLGGIWMHRRCKHRTGKADGLTVLWPHHQDFQSYPK

SEQ ID NO: 4 - DNA, 317 bp, Human
gggccacgccaggaatattcagaaaataatgagaactacattgaagtgccattgatttttgatcctgtcacaagagaggatttgcacatggattttaaatgtgttgtccataataccctgagttttcagacactacgcaccacagtcaaggaagcctcctccacgttctcctggggcattgtgctggccccactttcactggccttcttggttttggggggaatatggatgcacagacggtgcaaacacagaactggaaaagcagatggtctgactgtgctatggcctcatcatcaagactttcaatcctatcccaa

SEQ ID NO: 5 - DNA, 20 bp, Artificial Sequence
  left primer for mIL-1RII
tacgcaccacagtcaaggaa

SEQ ID NO: 6 - DNA, 20 bp, Artificial Sequence
  right primer for mIL-1RII
aagaaggccagtgaaagtgg

SEQ ID NO: 7 - DNA, 76 bp, Artificial Sequence
  amplicon for mIL-1RII
tacgcaccacagtcaaggaagcctcctccacgttctcctggggcattgtgctggccccactttcactggccttctt

SEQ ID NO: 8 - DNA, 449 bp, Human
atctcatacccgcaaattttaaccttgtcaacctctggggtattagtatgccctgacctgagtgaattcacccgtgacaaaactgacgtgaagattcaatggtacaaggattctcttcttttggataaagacaatgagaaatttctaagtgtgagggggaccactcacttactcgtacacgatgtggccctggaagatgctggctattaccgctgtgtcctgacatttgcccatgaaggccagcaatacaacatcactaggagtattgagctacgcatcaagaaaaaaaaagaagagaccattcctgtgatcatttcccccctcaagaccatatcagcttctctggggtcaagactgacaatcccgtgtaaggtgtttctgggaaccggcacacccttaaccaccatgctgtggtggacggccaatgacacccacatagagagcgccta

SEQ ID NO: 9 - DNA, 20 bp, Artificial Sequence
  left primer for sIL-1RII
ggggaccactcacttactcg

SEQ ID NO: 10 - DNA, 20 bp, Artificial Sequence
  right primer for sIL-1RII
cagcggtaatagccagcatc

SEQ ID NO: 11 - DNA, 60 bp, Artificial Sequence
  amplicon for sIL-1RII
ggggaccactcacttactcgtacacgatgtggccctggaagatgctggctattaccgctg

(19) United States
(12) Patent Application Publication
(10) Pub. No.: US 2010/0069343 A1
(43) Pub. Date: Mar. 18, 2010

(54) Methods of predicting steroid responsiveness with Il-1RII

(76) Inventor: Andrea Vambutus, Glen Head, NY (US)

Correspondence Address: AMSTER, ROTHSTEIN & EBENSTEIN LLP, 90 PARK AVENUE, NEW YORK, NY, 10016, US

(21) Appl. No.: 12/460,381

(22) Filed: Jul. 17, 2009

Related U.S. Application Data
(60) Provisional application No. 61/135,301, filed on Jul. 18, 2008.

(51) Int. Cl. A61K 31/56; C12Q 1/02; C12Q 1/68; C12Q 1/48; A61P 37/06; A61P 27/16

(52) U.S. Cl. 514/178; 435/29; 435/6; 435/15

(57) ABSTRACT

Methods are provided for determining in vitro whether or not a patient will respond to steroid treatment based on the levels of interleukin-1 receptor type II (IL-1RII) in a sample of mononuclear immune cells obtained from the patient before steroid treatment and/or on the change in IL-1RII in the patient's mononuclear immune cells in response to an in vitro steroid challenge.

### CROSS REFERENCE TO RELATED APPLICATION

This application is a U.S. Utility Application claiming the benefit of U.S. Provisional Application No. 61/135,301, filed Jul. 18, 2008, the content of which is hereby incorporated by reference in its entirety.

### FIELD OF THE INVENTION

The present invention generally relates to methods of determining whether or not a patient will respond to steroid treatment. More particularly, the invention relates to methods of determining whether or not a patient will respond to steroid treatment based on the level of interleukin-1 receptor type II (IL-1RII) in a sample of mononuclear immune cells obtained from the patient before steroid treatment and/or on the change in IL-1RII in the patient's mononuclear immune cells in response to an in vitro steroid challenge.

### BACKGROUND OF THE INVENTION

Throughout this application various publications are referred to in parenthesis. Citations for these references may be found at the end of the specification immediately preceding the claims. The disclosures of these publications are hereby incorporated by reference in their entireties into the subject application to more fully describe the art to which the subject application pertains.

Autoimmune inner ear disease (AIED) causes progressive sensorineural hearing loss as a result of either a primary autoimmune disorder of the inner ear or a systemic autoimmune disorder that secondarily affects the ear (Ruckenstein, 2004). Patients with AIED are treated with steroids, which is standard of care to maintain hearing during periods of sudden decline. However, for many of these patients whose hearing fluctuates frequently, steroid therapy invokes considerable risk, and those that initially respond may become refractory to treatment over time (Broughton et al., 2004). Other immunosuppressive treatments have not been successful, most notably, methotrexate (Harris et al., 2003). For AIED patients with progressive sensorineural hearing loss refractory to medical therapy who derive little to no benefit with hearing aids, cochlear implants are effective (Quaranta et al., 2002).

Sera from patients with AIED has been assayed for antibodies against various inner ear antigens and antibody responses to a number of proteins has been reported. Interestingly, a single target antigen has not been identified (as reviewed by Ryan et al., 2002). Numerous antigens in the inner ear have been identified in AIED patients including the 68 kD protein (Moscicki et al., 1994) and the PO protein (Cao et al., 1996). Unfortunately, seropositivity serves to assist diagnosis, but does not aid in development of antigen-specific treatment of these patients, and does not elucidate mechanism.

A limitation in determining a more appropriate medical therapy for AIED patients with residual hearing has been the inability to further characterize the mechanism of inflammation and autoimmune destruction that occurs. This is due in large part to the lack of access to the cochlea in humans. To this end, animal models of autoimmune hearing loss have been used. A connection between the inner ear and lymphatic system has been shown in guinea pigs, providing evidence that immune cells can access the inner ear from the peripheral circulation (as reviewed by Harris et al., 2003 and Gloddek and Arnold, 2002). In antigenically challenged sensitive animals, radiolabeled lymphocytes were identified in the scala tympani of the cochlea (Gloddek and Arnold, 2002). Additionally, T cells from the systemic circulation proliferate in the endolymphatic sac (Iwai et al., 1999). Proteins in the perilymph can reach immune cells in the endolymphatic sac (Yeo et al., 1995). Serum antibodies have been shown to be transferred to the perilymph is a chinchilla model as well (Mogi et al., 1985). Systemic autoimmune models, such as the mouse model for lupus, have characteristic hearing loss. These animals, like humans with AIED, are steroid responsive (Trune et al., 1999).

The murine autoimmune model also highlights the potential contribution of peripheral blood mononuclear cells (PBMC) in the pathogenesis of autoimmune hearing loss. In animals with advanced AIED, significant breakdown of the blood-endolymph barrier was observed (Trune et al., 1999; Lin and Trune, 1997). In contrast, before the onset of disease occurred, the integrity of the barrier was maintained (Lin and Trune, 1997). These studies reflect the potential for systemic PBMC dysfunction in AIED, but this has not been clinically validated.

Recent animal studies highlighted the role of inflammatory mediators in the inner ear in response to antigen. Tumor necrosis factor-alpha (TNF-α) release occurs after administration of antigen, and can be reversed by the administration of Etanercept (Satoh et al., 2002), suggesting the possible therapeutic role for TNF antagonists in the treatment of AIED. In addition, the role of interleukin-1 beta (IL-1β) and the innate immune response have been shown to be potential critical mediators in adaptive immune responses in the cochlea (Hashimoto et al., 2005).

Accordingly, a method is needed that can be used to determine whether a patient with an autoimmune disease or disorder, such as AIED, will respond to steroid treatment, without unnecessarily subjecting the patient to the risks of steroid treatment.

### SUMMARY OF THE INVENTION

The present invention is directed to a method of determining whether or not a patient will respond to steroid therapy, the method comprising in vitro treatment of mononuclear immune cells obtained from the patient with a glucocorticoid; in vitro measurement of membrane-bound interleukin-1 receptor type II (mIL-1RII) levels in a sample of the patient's mononuclear immune cells before and after in vitro treatment with the glucocorticoid; and comparison of the mIL-1RII levels in the cells before and after the in vitro glucocorticoid treatment, wherein an increase in mIL-1RII levels after in vitro glucocorticoid treatment that is greater than the response observed from mononuclear immune cells of non-responding subjects indicates that the patient will respond to steroid therapy, or wherein a change in mIL-1RII levels after glucocorticoid treatment that is not greater than a response observed from mononuclear immune cells of non-responding subjects indicates that the patient will not respond to steroid treatment.

The invention also provides a method of determining whether or not a patient will respond to steroid treatment, the method comprising measuring for basal membrane-bound interleukin-1 receptor type II (mIL-1RII) in a sample of the patient's mononuclear immune cells, wherein a failure to detect mIL-1RII indicates the patient will respond to steroid treatment, or wherein detection of a level of mIL-1RII below the level from mononuclear immune cells of subjects who are known to not respond to steroid treatment indicates the patient will respond to steroid treatment.

In addition, the present invention provides a method for treating a subject having autoimmune inner ear disease, sudden sensorineural hearing loss, Ménière's disease, or acoustic trauma comprising administering to the subject an effective amount of an IL-1 inhibitor or antagonist.

### BRIEF DESCRIPTION OF THE DRAWINGS

FIG. 1A. Comparison of RNA from cultured PBMC of controls (CTRL) and AIED either unstimulated (UNTX), stimulated with 23 valent pneumococcal vaccine (PNEUMO) or autologous cochlear perilymph (C) and compared by U133A 2.0 Affymetrix chip analysis (Genesifter): with a threshold of 2 and a Benjamini & Hochberg correction. Only 10 genes were significantly different, only one of them unique to the cochlear fluid-stimulated condition IL-1RII (affy ID 205403), P<0.05.

FIG. 1B is a graph of the microarray results for IL-1RII. Analysis demonstrated a 4.9-fold increase in controls versus AIED patients in IL-1RII decoy receptor levels in response to cochlear fluid, p<0.05. PBMC from controls or AIED patients were cultured with either no stimulus (white bar), pneumococcal vaccine (grey bar) or the patient's own cochlear fluid (black bar).

FIG. 2A is graph of the results of Quantitative Real Time Polymerase Chain Reaction (Q-RT-PCR) for IL-1RII. Q-RT-PCR was performed to confirm differences in RNA expression seen in the microarray results. RNA was available from 3 AIED patients and 2 controls. Fold change in IL-1RII levels of cochlear-stimulated over unstimulated was calculated for both patient groups and compared. Controls had a 5.0 fold increase compared to AIED patients in IL-1RII levels (p=0.03 by an unpaired t-test).

FIG. 2B is a graph of the results of Enzyme-Linked ImmunoSorbent Assay (ELISA) for soluble IL-1RII. Supernatants from PBMC of 1 AIED patient and 1 control were used to determine the level of soluble receptor. The results shown are after 16 hours of stimulation. Stimulus conditions were either no stimulus (white bar), pneumococcal stimulus (grey bar) or autologous cochlear fluid (black bar).

FIG. 3. Q-RT-PCR for 3 control subjects and 5 AIED subjects comparing autologous perilymph stimulated to unstimulated PBMC for both the shorter soluble IL-1RII (sIL-1RII) and the longer membrane bound form of IL-1RII (mIL-1RII). Control subjects, but not those with AIED, respond to autologous perilymph stimulation with markedly increased expression of mIL-1RII. These differences were statistically significant for mIL-1RII using a Mann Whitney test (p=0.03).

FIG. 4. ELISA for soluble IL-1RII (sIL-1RII). Supernatants from 16 hour PBMC cultures from 3 AIED patients and 2 controls were used to determine the level of soluble receptor. Stimulus conditions were no stimulus (white bar), pneumococcal stimulus (grey bar) or autologous cochlear perilymph (black bar). Relative amounts were set at one for the unstimulated condition as this data set included 2 children (one AIED, one control). sIL-1RII levels are less in children; however, a similar patterns of expression were observed. Standard deviations are shown.

FIG. 5A. 10 patients with presumptive AIED and sudden declines in hearing were enrolled, blood samples were taken and then the patients were treated with 60 mg prednisone/day×7 days and then tapered. Post-treatment audiograms were obtained between 7-14 days later. Improvement (Δ) was measured as the average of 500, 1, 2, 4 & 8 kHz. Responders (R) all returned to prior baseline hearing. The patient that improved 13.3 dB represented a patient that returned to normal hearing thresholds. PBMC were isolated, divided and cultured for 16 hours with either 0 μg/ml or increasing concentrations of dexamethasone (8, 20, 40 or 80 μg/ml). Fold changes in levels of mIL-1RII by Q-RT-PCR after in vitro challenge with dexamethasone (expressed as fold change over baseline) predicted steroid responsiveness in AIED. In data on 7 subjects, the fold-increase was log-transformed in order to better meet the assumptions of a normal distribution with equal variance. This analysis showed that mean (log) fold change was significantly higher in responders (R) than non-responders (NR) (P<0.0001, t-test).

FIG. 5B. Pre- and post-treatment mIL-1RII levels in a subset of clinical responders and non-responders. Responders have essentially zero basal mIL-1RII expression, compared with non-responders that have substantially higher basal levels. Although both responders and non-responders demonstrate increased expression in response to in vitro dexamethasone stimulation, the fold change for responders is far larger than for non-responders, mainly attributable to the very low basal expression of mIL-1RII in cultures of PBMCs obtained from responders before the course of prednisone therapy.

### DETAILED DESCRIPTION OF THE INVENTION

The invention is directed to methods of determining whether or not a patient will respond to steroid treatment. The methods are particularly useful for patients who have an autoimmune disease or disorder for which steroid treatment is being considered. The autoimmune disease or disorder may be inflammatory bowel disease, such as ulcerative colitis, rheumatoid arthritis, systemic lupus erythematosus, asthma, autoimmune inner ear disease (AIED), transplant rejection, such as renal transplant rejection, or other autoimmune conditions.

The invention provides a method of determining whether or not a patient will respond to steroid treatment, the method comprising:

(a) treating mononuclear immune cells from the patient with a glucocorticoid;

(b) measuring membrane-bound interleukin-1 receptor type II (mIL-1RII) levels in a sample of the patient's mononuclear immune cells before and after treatment with the glucocorticoid; and

(c) comparing the mIL-1RII levels in the cells before and after glucocorticoid treatment,

wherein an increase in mIL-1RII levels after glucocorticoid treatment above a response observed from mononuclear immune cells of non-responding subjects indicates that the patient will respond to steroid treatment, or

wherein a change in mIL-1RII levels after glucocorticoid treatment that is not greater than a response observed from mononuclear immune cells of non-responding subjects indicates that the patient will not respond to steroid treatment.

Whether or not a change in mIL-1RII levels after glucocorticoid treatment constitutes an increase in mIL-1RII levels that is indicative of whether or not the patient will respond to steroid treatment can be determined by comparing the change observed in samples from the patient to the change observed from mononuclear immune cells of subjects who are known to respond to steroid treatment and/or who are known not to respond to steroid treatment.

Alternatively, or in addition, the change in mIL-1RII levels after glucocorticoid treatment can be evaluated directly by the amount of the change observed in samples from the patient. For example, an increase in mIL-1RII levels in the mononuclear immune cells after glucocorticoid treatment of at least 10,000-fold indicates that the patient will respond to steroid treatment. Preferably, the increase is least 100,000-fold, and more preferably at least 1,000,000 fold. Increases of this magnitude are known to be above the response observed from mononuclear immune cells of subjects who do not respond to steroid treatment. Conversely, an increase in mIL-1RII levels in the cells after glucocorticoid treatment of 100-fold or less, or 500-fold or less, or 1,000-fold or less, indicates that the patient not will respond to steroid treatment. Such changes in mIL-1RII levels after glucocorticoid treatment are within the responses observed from mononuclear immune cells of subjects who do not respond to steroid treatment.

The invention also provides a method of determining whether or not a patient will respond to steroid treatment, the method comprising measuring for basal membrane-bound interleukin-1 receptor type II (mIL-1RII) in a sample of the patient's mononuclear immune cells, wherein a failure to detect mIL-1RII indicates the patient will respond to steroid treatment, or wherein detection of a level of mIL-1RII below the level from mononuclear immune cells of subjects who are known to not respond to steroid treatment indicates that the patient will respond to steroid treatment. For example, where more than 35 cycles, or more than 40 cycles, or more than 45 cycles, of quantitative real-time polymerase chain reaction (Q-RT-PCR) are required to detect mIL-1RII, or fail to detect mIL-1RII, the method indicates that the patient will respond to steroid treatment.

The mononuclear cells can be peripheral blood mononuclear cells (PMBC).

The steroid used in the treatment of the mononuclear cells can be a glucocorticoid. The glucocorticoid can be any of the forms of glucocorticoid known in the art, including, but not limited to, hydrocortisone (cortisol), prednisone, prednisolone, methylprednisolone, dexamethasone, betamethasone, triamcinolone, beclometasone and fludrocortisone acetate. Preferably, the glucocorticoid is a synthetic glucorticoid, such as dexamethasone.

IL-1RII protein binds interleukin alpha (IL1A), interleukin beta (IL1B), and interleukin 1 receptor, type I (IL1R1/IL1RA), and acts as a decoy receptor that inhibits the activity of its ligands. Interleukin 4 (IL4) is reported to antagonize the activity of interleukin 1 by inducing the expression and release of this cytokine. This gene and three other genes form a cytokine receptor gene cluster on chromosome 2q12. Two alternatively spliced transcript variants encoding the same protein have been reported (IL1R2 interleukin 1 receptor, type II [Homo sapiens], updated 2 Dec. 2007, http://www.ncbi.nlm.nih.gov/sites/entrez?Db=gene&Cmd=ShowDetailView&TermToSearch=7850#refseq).

Preferably, the human interleukin 1 receptor, type II nucleic acid has one of the following variant sequences:

```
(SEQ ID NO: 1)
      1 cccgtgagga ggaaaaggtg tgtccgctgc cacccagtgt gagcgggtga caccacccgg
    61 ttaggaaatc ccagctccca agagggtata aatccctgct ttactgctga gctcctgctg
  121 gaggtgaaag tctggcctgg cagccttccc caggtgagca gcaacaaggc cacgtgctgc
  181 tgggtctcag tcctccactt cccgtgtcct ctggaagttg tcaggagcaa tgttgcgctt
  241 gtacgtgttg gtaatgggag tttctgcctt cacccttcag cctgcggcac acacaggggc
  301 tgccagaagc tgccggtttc gtgggaggca ttacaagcgg gagttcaggc tggaagggga
  361 gcctgtagcc ctgaggtgcc cccaggtgcc ctactggttg tgggcctctg tcagcccccg
  421 catcaacctg acatggcata aaaatgactc tgctaggacg gtcccaggag aagaagagac
  481 acggatgtgg gcccaggacg gtgctctgtg gcttctgcca gccttgcagg aggactctgg
  541 cacctacgtc tgcactacta gaaatgcttc ttactgtgac aaaatgtcca ttgagctcag
  601 agtttttgag aatacagatg ctttcctgcc gttcatctca tacccgcaaa ttttaacctt
  661 gtcaacctct ggggtattag tatgccctga cctgagtgaa ttcacccgtg acaaaactga
  721 cgtgaagatt caatggtaca aggattctct tcttttggat aaagacaatg agaaatttct
  781 aagtgtgagg gggaccactc acttactcgt acacgatgtg gccctggaag atgctggcta
  841 ttaccgctgt gtcctgacat ttgcccatga aggccagcaa tacaacatca ctaggagtat
  901 tgagctacgc atcaagaaaa aaaaagaaga gaccattcct gtgatcattt cccccctcaa
  961 gaccatatca gcttctctgg ggtcaagact gacaatcccg tgtaaggtgt ttctgggaac
1021 cggcacaccc ttaaccacca tgctgtggtg gacggccaat gacacccaca tagagagcgc
1081 ctacccggga ggccgcgtga ccgaggggcc acgccaggaa tattcagaaa ataatgagaa
1141 ctacattgaa gtgccattga tttttgatcc tgtcacaaga gaggatttgc acatggattt
1201 taaatgtgtt gtccataata ccctgagttt tcagacacta cgcaccacag tcaaggaagc
1261 ctcctccacg ttctcctggg gcattgtgct ggccccactt tcactggcct tcttggtttt
1321 ggggggaata tggatgcaca gacggtgcaa acacagaact ggaaaagcag atggtctgac
1381 tgtgctatgg cctcatcatc aagactttca atcctatccc aagtgaaata aatggaatga
1441 aataattcaa acacaaaaaa aaaaaaaaaa aaaaaaaaaa aaaa,
```

GenBank Accession No. BC039031, version BC039031.1 GI:24660342, Strausberg et al. (2002); and

```
(SEQ ID NO: 2)
      1 gggatgggag atactgttgt ggtcacctct ggaaaataca ttctgctact cttaaaaact
    61 agtgacgctc atacaaatca acagaaagag cttctgaagg aagactttaa agctgcttct
  121 gccacgtgct gctgggtctc agtcctccac ttcccgtgtc ctctggaagt tgtcaggagc
  181 aatgttgcgc ttgtacgtgt tggtaatggg agtttctgcc ttcacccttc agcctgcggc
  241 acacacaggg gctgccagaa gctgccggtt tcgtgggagg cattacaagc gggagttcag
  301 gctggaaggg gagcctgtag ccctgaggtg cccccaggtg ccctactggt tgtgggcctc
  361 tgtcagcccc cgcatcaacc tgacatggca taaaaatgac tctgctagga cggtcccagg
  421 agaagaagag acacggatgt gggcccagga cggtgctctg tggcttctgc cagccttgca
  481 ggaggactct ggcacctacg tctgcactac tagaaatgct tcttactgtg acaaaatgtc
  541 cattgagctc agagtttttg agaatacaga tgctttcctg ccgttcatct catacccgca
  601 aattttaacc ttgtcaacct ctggggtatt agtatgccct gacctgagtg aattcacccg
  661 tgacaaaact gacgtgaaga ttcaatggta caaggattct cttcttttgg ataaagacaa
  721 tgagaaattt ctaagtgtga gggggaccac tcacttactc gtacacgatg tggccctgga
  781 agatgctggc tattaccgct gtgtcctgac atttgcccat gaaggccagc aatacaacat
  841 cactaggagt attgagctac gcatcaagaa aaaaaaaaga agagaccatt cctgtgatca
  901 tttcccccct caagaccata tcagcttctc tggggtcaag actgacaatc ccgtgtaagg
  961 tgtttctggg aaccggcaca cccttaacca ccatgctgtg gtggacggcc aatgacaccc
1021 acatagagag cgcctacccg ggaggccgcg tgaccgaggg gccacgccag gaatattcag
1081 aaaataatga gaactacatt gaagtgccat tgatttttga tcctgtcaca agagaggatt
1141 tgcacatgga ttttaaatgt gttgtccata ataccctgag ttttcagaca ctacgcacca
1201 cagtcaagga agcctcctcc acgttctcct ggggcattgt gctggcccca ctttcactgg
1261 ccttcttggt tttgggggga atatggatgc acagacggtg caaacacaga actggaaaag
1321 cagatggtct gactgtgcta tggcctcatc atcaagactt tcaatcctat cccaagtgaa
1381 ataaatggaa tgaaataatt caaaaaaaaa aaaaaaaaaa aaaaaaaaaa,
```

GenBank Accession No. BC012346, version BC012346.1 GI:15214437, Strausberg et al. (2002).

Preferably, the human interleukin 1 receptor, type II protein has the sequence:

```
(SEQ ID NO: 3)
    1 mirlyvlvmg vsaftlqpaa htgaarscrf rgrhykrefr legepvalrc pqvpywlwas
  61 vsprinltwh kndsartvpg eeetrmwaqd galwllpalq edsgtyvctt rnasycdkms
121 ielrvfentd aflpfisypq iltlstsgvl vcpdlseftr dktdvkigwy kdsllldkdn
181 ekflsvrgtt hllvhdvale dagyyrcvlt fahegqqyni trsielrikk kkeetipvii
241 splktisasl gsrltipckv flgtgtpltt mlwwtandth iesaypggrv tegprqeyse
301 nnenyievpl ifdpvtredl hmdfkcvvhn tlsfqtlrtt vkeasstfsw givlaplsla
361 flviggiwmh rrckhrtgka dgltvlwphh qdfqsypk,
```

GenBank Accession No. AAH39031, version AAH39031.1 GI:24660343, Strausberg et al. (2002).

The IL-1RII can be membrane-bound IL-1RII (mIL-1RII) or soluble IL-1RII (s IL-1RII). Preferably, the IL-1RII is membrane-bound IL-1RII (mIL-1RII).

The membrane-bound (mIL-1RII) microarray sequence (SEQ ID NO:4) is:

gggccacgccaggaatattcagaaaataatgagaactacattgaagtgcc
attgatttttgatcctgtcacaagagaggatttgcacatggattttaaat
gtgttgtccataataccctgagttttcagacactacgcaccacagtcaag
gaagcctcctccacgttctcctggggcattgtgctggccccactttcact
ggccttcttggttttggggggaatatggatgcacagacggtgcaaacaca
gaactggaaaagcagatggtctgactgtgctatggcctcatcatcaagac
tttcaatcctatcccaa.

For the membrane-bound (mIL-1RII), Q-RT-PCR primers/amplicon:

Use Universal ProbeLibrary probe: #2, cat.no.
04684982001 (Formerly Exiqon ProbeLibrary probe:
Human #02)

| Primer | Length | Position | Tm | % GC | Sequence |
|---|---|---|---|---|---|
| Left | 20 | 134-153 | 60 | 50 | tacgcaccacagtcaaggaa |
| Primer | | | | | (SEQ ID NO: 5) |
| Right | 20 | 190-209 | 59 | 50 | aagaaggccagtgaaagtgg |
| Primer | | | | | (SEQ ID NO: 6) |

Amplicon (76 nt)

tacgcaccacagtcaaggaagcctcctccacgttctcctggggcattgtg
ctggccccactttcactggccttctt
(SEQ ID NO: 7)

The soluble (sIL-1RII) microarray sequence (SEQ ID NO:8) is:

atctcatacccgcaaattttaaccttgtcaacctctggggtattagtatg
ccctgacctgagtgaattcacccgtgacaaaactgacgtgaagattcaat
ggtacaaggattctcttcttttggataaagacaatgagaaatttctaagt
gtgagggggaccactcacttactcgtacacgatgtggccctggaagatgc
tggctattaccgctgtgtcctgacatttgcccatgaaggccagcaataca
acatcactaggagtattgagctacgcatcaagaaaaaaaaagaagagacc
attcctgtgatcatttcccccctcaagaccatatcagcttctctggggtc
aagactgacaatcccgtgtaaggtgtttctgggaaccggcacacccttaa
ccaccatgctgtggtggacggccaatgacacccacatagagagcgccta.

For the soluble (sIL-1RII), Q-RT-PCR primers/amplicon:

Use Universal ProbeLibrary probe: #81, cat.no.
04689046001 (Formerly Exiqon ProbeLibrary probe:
Human #81)

| Primer | Length | Position | Tm | % GC | Sequence |
|---|---|---|---|---|---|
| Left | 20 | 156-175 | 59 | 60 | ggggaccactcacttactcg |
| Primer | | | | | (SEQ ID NO: 9) |
| Right | 20 | 196-215 | 60 | 55 | cagcggtaatagccagcatc |
| Primer | | | | | (SEQ ID NO: 10) |

Amplicon (60 nt)

ggggaccactcacttactcgtacacgatgtggccctggaagatgctggct
attaccgctg
(SEQ ID NO: 11)

The IL-1RII levels can be measured by determining the IL-1RII mRNA or protein levels in the cells. The levels of IL-1RII can be detected by detection methods readily determined from the known art, including, without limitation, immunological techniques such as Western blotting, hybridization analysis, fluorescence imaging techniques, and/or radiation detection. For example, the IL-1RII mRNA levels can be determined by performing Quantitative Real Time Polymerase Chain Reaction (Q-RT-PCR), and the IL-1RII protein levels can be determined for example by performing Enzyme-Linked ImmunoSorbent Assay (ELISA).

IL-1RII levels can be assayed using an agent that specifically binds IL-1 Rn such as, for example, an antibody, a peptide or an aptamer. As used herein, the term “antibody” encompasses whole antibodies and fragments of whole antibodies wherein the fragments specifically bind to IL-1RII. Antibody fragments include, but are not limited to, F(ab′)₂ and Fab′ fragments and single chain antibodies. F(ab′)₂ is an antigen binding fragment of an antibody molecule with deleted crystallizable fragment (Fc) region and preserved binding region. Fab′ is ½ of the F(ab′)₂ molecule possessing only ½ of the binding region. The term antibody is further meant to encompass polyclonal antibodies and monoclonal antibodies. Antibodies may be produced by techniques well known to those skilled in the art. The antibody can be, e.g., any of an IgA, IgD, IgE, IgG, or IgM antibody. Aptamers are single stranded oligonucleotides or oligonucleotide analogs that bind to a particular target molecule, such as a protein. Thus, aptamers are the oligonucleotide analogy to antibodies. Both RNA and single stranded DNA (or analog) aptamers can be used.

The agent that specifically binds to IL-1RII can be labeled with a detectable marker. Labeling may be accomplished using one of a variety of labeling techniques, including peroxidase, chemiluminescent, and/or radioactive labels known in the art. The detectable marker may be, for example, a nonradioactive or fluorescent marker, such as biotin, fluorescein (FITC), acridine, cholesterol, or carboxy-X-rhodamine, which can be detected using fluorescence and other imaging techniques readily known in the art. Alternatively, the detectable marker may be a radioactive marker, including, for example, a radioisotope. The radioisotope may be any isotope that emits detectable radiation, such as, for example, ³⁵S, ³²P, or ³H. Radioactivity emitted by the radioisotope can be detected by techniques well known in the art. For example, gamma emission from the radioisotope may be detected using gamma imaging techniques, particularly scintigraphic imaging.

IL-1RII levels may be detected through hybridization analysis of nucleic acid using one or more nucleic acid probes which specifically hybridize to IL-1M′ mRNA. The nucleic acid probes may be DNA or RNA, and may vary in length from about 8 nucleotides to the entire length of the nucleic acid. Hybridization techniques are well known in the art. The probes may be prepared by a variety of techniques known to those skilled in the art, including, without limitation, restriction enzyme digestion, and automated synthesis of oligonucleotides using commercially-available oligonucleotide synthesizers.

The nucleic acid probes may be labeled with one or more detectable markers. Labeling of the nucleic acid probes may be accomplished using a number of methods known in the art (e.g., nick translation, end labeling, fill-in end labeling, polynucleotide kinase exchange reaction, random priming, or SP6 polymerase) with a variety of labels (e.g., radioactive labels, such as ³⁵S, ³²P, or ³H, or nonradioactive labels, such as biotin, fluorescein (FITC), acridine, cholesterol, or carboxy-X-rhodamine (ROX)).

The IL-1RII mRNA or protein levels can be compared to the levels of a control mRNA or protein, such as, for example, β-actin.

The present invention also provides a method for treating a subject having autoimmune inner ear disease, sudden sensorineural hearing loss, Ménière's disease, or acoustic trauma comprising administering to the subject an effective amount of an IL-1 inhibitor or antagonist. As used herein, an “effective amount” is preferably an amount of the IL-1 inhibitor or antagonist effective to treat autoimmune inner ear disease, sudden sensorineural hearing loss, Ménière's disease, or acoustic trauma, and/or symptoms associated with these conditions. The prototypical IL-1 inhibitor/antagonist is “anakinra”, an interleukin-1 (IL-1) receptor antagonist marketed under the tradename “Kineret” (Amgen). The anakinra molecule is a recombinant, non-glycosylated version of human IL-1RA (RA for receptor antagonist), which is another example of a IL-1 inhibitor/antagonist that can be used in the method of the present invention. Anakinra differs from native human IL-1 RA by the addition of a single methionine residue at the amino terminus. Other examples of IL-1 inhibitors/antagonists that can be used in accordance with the method of the present invention are described in U.S. Pat. No. 7,087,224, which is specifically incorporated by reference herein.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

### Example Summary

Autoimmune Inner Ear Disease (AIED) is poorly characterized clinically and molecularly. Hearing loss can be profound, requiring a cochlear implant. There has been no common biomarker that can be used to definitively identify AIED. All patients suspected of having AIED are given glucocorticoids during periods of acute hearing loss, but only half initially respond, and still fewer remain responsive over time. The present study has identified a novel biomarker of steroid responsive AIED, specifically the Interleukin-1 Receptor type 2 (IL-1RII). IL-1RII is a molecular decoy that traps interleukin-10 (IL-1β), and thereby suppresses inflammation induced by IL-1β signaling. Early in the course of AIED in patients who will respond favorably to steroid therapy, the membrane bound form of IL-1M (mIL-1RII) is not expressed in the peripheral blood mononuclear cells (PBMCs). However, mIL-1RII is strongly induced when cultures of these PBMCs are challenged in vitro with a glucocorticoid such as dexamethasone. The degree of pre-treatment in vitro induction is predictive of clinical audiometric responsiveness (P<0.0001). In contrast, clinical steroid non-responsive patients suspected of having AIED express significantly higher basal levels of mIL-1RII in their cultured PBMCs, yet the cultured PBMCs show only minimal enhancement in the expression of mIL-1RII in response to in vitro steroid challenge.

### Methods

Cochlear Implant Subjects. All patients met current criteria for cochlear implantation and had a bilateral profound sensorineural hearing loss (SNHL). All patients with AIED had periods of rapid hearing loss and were no longer steroid responsive. The majority of those with AIED also had systemic autoimmune disease, thereby increasing the probability of inner ear involvement. Interestingly, none of these subjects had the same autoimmune disorder, discounting the possibility of a single class II HLA allele as the mediating factor. All adult controls had stable SNHL for over 30 years of clear, non-immunologic origin. This study was reviewed and approved by the Institutional Review Board of the North Shore-LIJ Health System. Nine patients were recruited (7 adults, 2 children) representing 4 controls and 5 with AIED. Their clinical demographics shown in Table 1.

At the time of cochlear implant surgery; blood was drawn to isolate PBMC. Perilymph fluid was harvested from the cochleostomy in a bloodless field. All patients enrolled have an excellent aided benefit with their cochlear implant, suggesting that no untoward effect of perilymph sampling was incurred. In order to control for a cochlear perilymph specific response, unstimulated PBMC were compared to cochlear perilymph stimulated PBMC and to pneumococcal stimulated PBMC, from both AIED and control patients. Three microarrays were performed per study subject: 1) perilymph+autologous PBMC, 2) pneumococcal antigen+autologous PBMC, and 3) PBMC alone; and administration of pneumococcal vaccine (23 valent pneumococcal vaccine (Pneumovax® (Merck)) 2 weeks prior to cochlear implant surgery, considered to be standard of care prior to cochlear implantation, served as a positive control.

TABLE 1
Clinical History of AIED and Control Patients

| | | Autoimmune | Hearing | PTA & | |
|---|---|---|---|---|---|
| Patient | Status | History | History | HINT | Serology |
| 40 y/o | Control | None | Congenital | PTA 100dB; | NP* |
| female^{1,2} | (birth | | loss - non | HINT 60dB | |
| | anoxia?) | | progressive | quiet: 0% | |
| 49 y/o male^{1,2} | Control | None | 40+ years loss | PTA 100dB; | NP* |
| | | | | HINT 50dB | |
| | | | | quiet: 10% | |
| 79 y/o | Control | None | 70+ years | PTA 110; | NP* |
| female^{1,3} | (LVA) | | LVA | HINT 55dB | |
| | | | | quiet: 0%; | |
| 1.5 y/o female³ | Control | None | Congenital - | PTA: NR at | NP* |
| | (Cx 26) | | stable | 102dB by | |
| | | | | ABR; IT- | |
| | | | | MAIS: 12% | |
| 36 y/o | AIED, | Rheumatoid | 29 years, | PTA 120dB; | P0, Rheumatoid factor IgM |
| female^{1,2} | progressive | Arthritis | periods of | HINT: 55dB | (14.2) & IgG (25.3), immune |
| | loss | | rapid loss | quiet: 0% | complex, ANA 1:1280 (HEp- |
| | | | | | 2), IgM phospholipids (10.9), |
| | | | | | immune complex (26.1) |
| 56 y/o male^{1,2} | AIED, | Type I IDDM | 2 years, rapid | PTA 120; | ANA 1:1280 (mouse kidney), |
| | rapid loss | | loss | HINT 70dB | ANA 1:320 (HEp-2), weakly |
| | | | | quiet: 0% | positive IgM phospholipid Ab |
| | | | | | (12.1) |
| 58 y/o | AIED, | Hashimoto's | 4 years, rapid | PTA 110dB; | Borderline for immune |
| female^{1,2,3} | rapid loss | thyroiditis | loss | HINT at | complex (21.4), weakly |
| | | | | 50dB quiet: | positive IgM phospholipids |
| | | | | 0% | (11.7), borderline rheumatoid |
| | | | | | factor IgG (20.8) |
| 51 y/o | AIED, | None | Sudden loss | PTA 80dB; | 68 kD positive |
| female^{2,3} | progressive | | left ear 10 | HINT 65dB | |
| | loss | | years prior, | quiet: 19% | |
| | | | sudden loss | | |
| | | | right ear <6 | | |
| | | | months | | |
| 3 y/o male³ | AIED, b/l | None | 6 months, | PTA 120dB; | Negative |
| | rapid | | rapid loss | IT-MAIS | |
| | SNHL | | | 8% | |

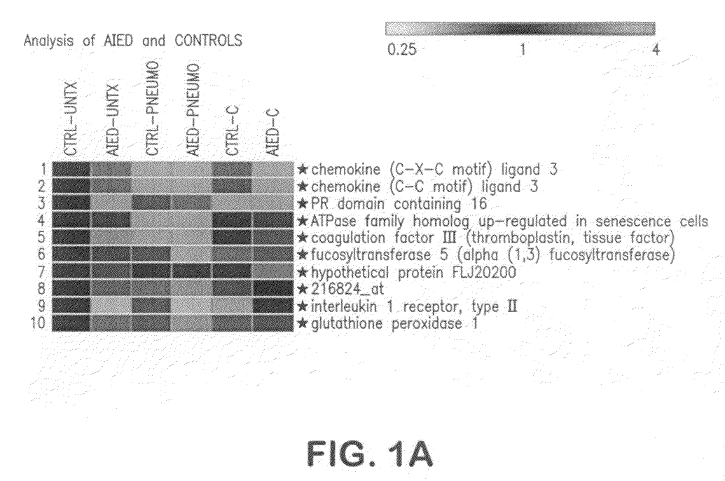

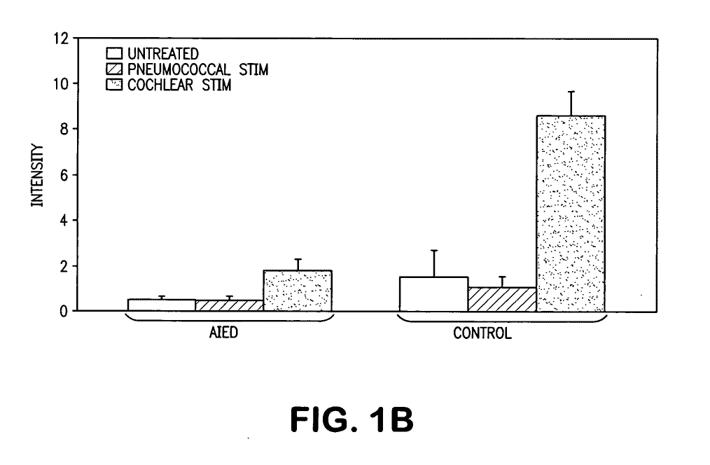

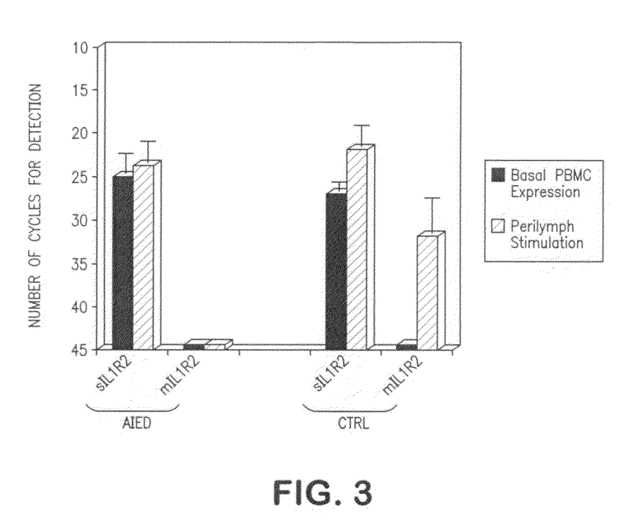

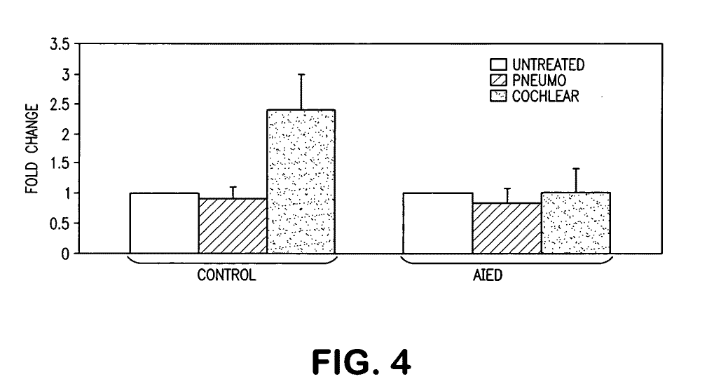

PTA: Pure tone average of the audiogram at 500, 1000, 2000 and 4000 Hz, (although 4000 Hz is not traditionally included, this information is important to speech perception for cochlear implantees and therefore PTA represents these four frequencies for these patients).
NP: Not Performed: control subjects did not have serology for AIED performed as there was no clinical indication for such studies,
HINT: Hearing in Noise Test;
IT-MAIS: Infant-Toddler Meaningful Auditory Integration Scale,
LVA: Large Vestibular Aqueduct;
Cx26: connexin 26 gene mutation.
Superscript numbers in column 1 refer to inclusion of the patient in data sets for the below FIGS.: 1: included in analysis for FIG. 1; 2: included in analysis for FIG. 3; 3: included in analysis of FIG. 4.

Prospectively Enrolled, Steroid Treated Subjects. Ten patients with AIED who had not undergone cochlear implantation were studied in a separate IRB approved protocol. They all had a clinical history suggestive of AIED, and met criteria defined for AIED trials (Mantovani et al. 1998). At the time of enrollment, they all demonstrated sudden declines in their hearing and were treated with 60 mg of prednisone daily for 7 days with a variable taper thereafter. The responders were defined as a 10 dB or greater average improvement at 250, 500, 1000, 2000, 4000 and 8000 Hz. All patients recruited must have had prior audiograms demonstrating their baseline hearing threshold. Furthermore, SNHL of greater than 30 dB at one or more frequencies in both ears with evidence of active deterioration (elevated threshold) in at least one ear of 15 dB at one frequency (excluding 250 or 8 kHz as a sole indicator), or 10 dB at 2+ frequencies developing in >3 days but <90 days (Niparko et al. 2005, Yeom et al. 2003). If the hearing loss evolved in less than 3 days, prior similar hearing declines must have occurred or the patient must have a systemic autoimmune disorder AND the patient must meet the audiology criteria outlined above. All patients with retrocochlear (vestibular schwannoma or other internal auditory canal s) pathology), patients who received prednisone or other immunosuppressive therapy within 3 months, or patients with vestibular symptoms coinciding with periods of hearing fluctuation were all excluded from this study. All patients received a minimum of 60 mg of prednisone daily for 7 days with a variable taper thereafter.

Microarray Analysis. PBMC were collected in heparin, isolated over a Ficoll-hypaque gradient, divided into 5 ml cultures (2×10⁶ cells/ml) and cultured in RPMI+10% FCS for 16 hours at 37° C. with 5% CO₂ with one of 3 stimuli (1) without antigenic stimulus (untx), (2) 100 μl pneumococcal vaccine (Wuorimaa et al. 2001) as a positive control or (3) with 15 μlof autologous cochlear perilymph. RNA was isolated using an affinity spin column (Qiagen) and 5 μg reverse transcribed into double stranded cDNA (Invitrogen) incorporating a T7 RNA polymerase promoter. The cDNA was phenol extracted, ethanol precipitated and biotinlyated cRNA generated by in vitro transcription (ENZO). cRNA was purified on a spin column (Qiagen), quantitated, and 20 μg fragmented and hybridized to the Affymetrix HG U133A 2.0 array (Affymetrix). Data sets were normalized using RMA and an ANOVA analysis was performed on grouped arrays by condition with a Benjamini and Hochberg correction, and a threshold of 2.0 fold change (Genesifter, VixXlabs).

Q-RT-PCR. Quantitative Real Time RT-PCR (Q-RT-PCR) was performed on PBMC from patients using TaqMan chemistry. The relative abundance of IL1R2 mRNA for 2 membrane bound (mIL-1RII) and the soluble (sIL-1RII) coding regions associated with the 2 Affymetrix probes sets 211372 (sIL-1RII) and 205403 (mIL-1RID compared to β-actin was determined using the Eurogentec RTqPCR mastermix (Eurogentec, Belgium) and ABI PRISM 7700 Sequence Detection System. Membrane bound IL-1RII, as reflected by Affymetrix probe set 205403 was detected by primers nt 1239-1258, 1295-1314, and taqman probe 1271-1278, whereas the shorter soluble form (sIL-1RII) reflected by the Affymetrix probe 211372 and primers nt 790-830-849, and taqman probe 820-827. The alternative splice site described by Liu et al. (1996) changing GAA to TAA occurs at nt 1118. These primers were added at final concentration of 200 nM and 100 nM respectively to 50 ng of total RNA. The conditions were 48° C. for 30′, 95° C. for 10′ and 45 cycles of 95° C. for 15″ and 60° C. for 1′. Data was analyzed using Sequence Detection System software version 1.9.1. Results were expressed as Ct (Threshold cycle) values, which is inversely proportional to the starting template copy number. Relative abundance of IL1R2 in cochlear fluid stimulated cells was calculated compared to untreated control samples using delta delta Ct method (User Bulletin #2, Applied Biosystems Inc).

Enzyme-Linked ImmumoSorbent Assay (ELISA) Analysis. ELISA for soluble IL-1RII (sIL-1RID. Supernatants from 16 hour PBMC cultures from 3 AIED patients and 2 controls were used to determine the level of soluble receptor (See Table 1 for inclusion). ELISA was performed according to manufacturers' instructions (R&D Systems). Stimulus conditions were either pneumococcal stimulus as used above or autologous cochlear perilymph and compared to unstimulated PBMC. A large number of duplicate samples were run to ensure accuracy.

### Results

Reduced expression of IL-1RII is associated with AIED. Microarray analysis of stimulated PBMC from AIED and control patients demonstrated an extremely limited number of statistically significant changes in gene expression. The effect was compared of adding autologous perilymph to peripheral blood mononuclear cells (PBMC) in steroid refractory patients with AIED who underwent cochlear implantation (end stage disease) versus control patients undergoing implantation for longstanding, non-immunologic, stable, SNHL by microarray analysis. Results for each patient were compared to unstimulated PBMC and pneumococcal stimulated PBMC. The rationale for the pneumococcal stimulation is that standard of care for a patient undergoing cochlear implantation is pneumococcal vaccination to prevent meningitis. Thus, all patients were primed by vaccination using a 23-valent-pneumococcal vaccine (Pneumovax®). Microarray results for AIED and control subjects were compared by the stimulus used to activate the PBMC and compared to unstimulated PBMC (FIG. 1A). Of note, only 10 genes were differentially expressed in AIED patients (p<0.05). Of those 10 genes, only one, the interleukin-1 receptor type 2 (IL 1-RII) (Affymetrix ID 205403, nt 1104-1485 (long membrane bound form)), was differentially expressed when autologous perilymph was added to PBMC of control subjects as compared with AIED subjects (FIG. 1A). Analysis of the expected common targets including IL-1RI, IL-1β, IL-1RA, heat shock proteins (HSP) and TNF-α failed to show differential expression (not shown).

PBMC from patients with AIED had a surprisingly minimal expression of IL-1RII in response to cochlear fluid when compared to the robust levels in controls (4.9 fold greater, p<0.05 (FIG. 1B)). No change was observed in the pneumococcal stimulated samples, suggesting a cochlear fluid specific response.

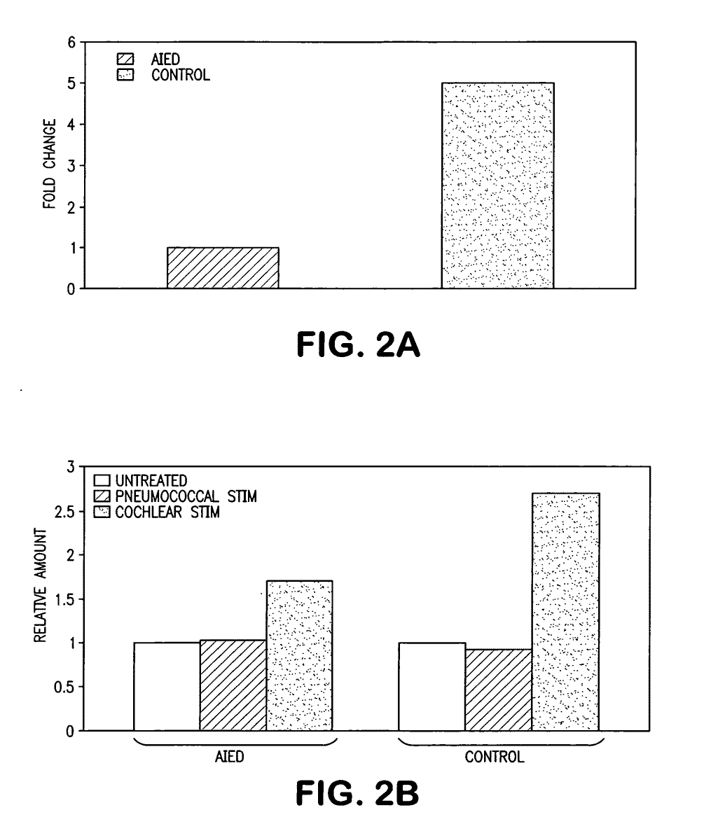

Q-RT-PCR confirmed the difference in IL-1RII levels in AIED and control patients (FIG. 2A), with a 5.0 fold greater mRNA level in control patients (p=0.03). More soluble IL-1RII protein was made by PBMC of control patients (FIG. 2B). Although differences were not as striking as RNA levels, this represents only soluble protein at a relatively early time point for changes in protein accumulation.

### Soluble and Membrane-Bound Forms of IL-1RII

RNA was examined using a quantitative real time polymerase chain reaction (Q-RT-PCR). IL-1RII exists in two forms: membrane-bound (mIL-1RII) and soluble (sIL-1RII). The RNA primers used detect the membrane bound, long form of the IL-1RII message, and were derived from the coding region of the IL-1RII gene on the Affymetrix gene array chip. The shorter soluble form is made by alternative splicing (Liu et al. 1996), or by aminopeptidase cleavage of the membrane bound protein. Interestingly, the shorter form is made by both AIED patients and controls, although at slightly lower levels in AIED subjects compared with controls (Affymetrix ID 211372, nt 635-1084) (FIG. 3). The longer, membrane bound form, (as detected by Affymetrix 205403 and Q-RT-PCR probes nt 1239-1258, 1295-1314, and taqman probe 1271-1278), is not expressed in unstimulated PBMC; however, in perilymph stimulated control PBMC, mIL-1RII is strongly induced. Just upstream of this Affymetrix probe is an alternative splice site at nt 1117 that introduces a premature stop codon (GAA→TAA) (Liu et al. 1996). The AIED patients do not express this long transcript (no transcript detected at 45 cycles). Notably, the inhibitory function of IL-1RII is selectively attributed to this long, membrane bound form (Neumann et al. 2000). Significant differences in the membrane bound IL-1RII mRNA between AIED and control groups were observed in the autologous perilymph stimulated PBMC (FIG. 3).

Soluble IL-1RII levels, which is likely reflective of the shorter IL-1Rn message (Affymetrix 211372), in AIED patients are also reduced in culture supernatants of PBMC stimulated with perilymph measured by ELISA (FIG. 4). This finding parallels the microarray results for Affymetrix probe 211372 and the Q-RT-PCR results for AIED and control subjects using primers derived from this region. This suggests that the significant alteration in AIED patients is in the membrane bound form of IL1R2; however, a smaller reduction in the soluble form exists in AIED patients.

### Membrane-Bound IL-1RII Levels Predict Steroid Responsive Sensorineural Hearing Loss (SNHL)

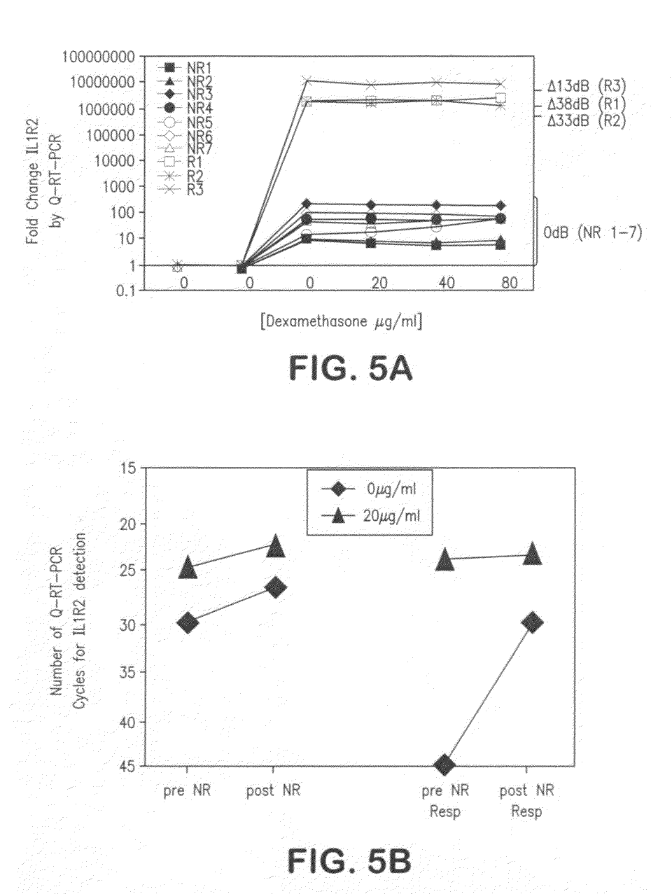

60% of patients studied by the AIED study group were steroid responsive (Niparko et al. 2005). In the present study, in vitro PBMC membrane-bound IL-IRII (mIL-1RII) mRNA expression in response to dexamethasone stimulation was compared to basal mIL-1RII expression in cultures of PBMCs collected prior to clinical steroid treatment. In addition, pre-treatment mIL-1RII expression was compared to post-treatment hearing recovery. Furthermore, pre- and post-treatment mIL-1RII expression were compared in a subset of clinical responders and non-responders. Pre-treatment PBMC cultures from clinical steroid responders demonstrated no basal IL-1RII expression; however, they dramatically augmented mIL-1RII expression in response to dexamethasone stimulation in vitro. All responders returned to their prior baseline hearing thresholds at the end of the corticosteroid treatment. Non-responders were defined as less than 5 dB improvement at the same frequencies or further decline of pure tone thresholds. Table 2 shows the clinical history of patients enrolled in this study. mIL-1RII levels were identified to be dramatically different in responders and non-responders (FIG. 5A). Of note, the dramatic difference in fold change of the corticosteroid responders is inversely proportional to basal mIL-1RII expression in PBMC (45 cycles, S.D. 0.0 in replicate samples) in an unstimulated condition, compared to corticosteroid non-responders (28.48 cycles, S.D.±3.34) (FIG. 5B, p<0.0001). In all patient samples, the unstimulated condition is the average of 2 independent cultured PBMC samples. Furthermore, post-steroid therapy mIL-1RII expression was determined in a subset of patients. Pre-treatment basal mIL-1RII expression in responders dramatically increased from no expression at 45 cycles to expression detected at 30 cycles post-treatment, compared with 30 cycles to 27 cycles post treatment in non-responders suggesting that the clinical response was a function of mIL-1RII expression. In contrast to mIL-1RII, levels of soluble IL-1RII were found to be not predictive.

TABLE 2
Clinical History of Patients

| | PRIOR | | | | AFFECT | | | | | | | AVG |
|---|---|---|---|---|---|---|---|---|---|---|---|---|
| PATIENT | TX? | PMH | SEROLOGY | MRI | EAR | 250 Hz | 500 Hz | 1000 Hz | 2000 Hz | 4000 Hz | 8000 Hz | Improve |
| 56WM | YES | NC | NA | NEG | PRETX | 35 | 45 | 60 | 105 | 120 | 105 | 0 dB |
| (NR1) | | | | | POSTTX | 40 | 45 | 60 | 105 | 120 | 105 | |
| 42 WF^{B} | YES | Hypo- | ANA 1:320, | NEG | PRETX | 50 | 75 | 90 | 95 | 95 | 85 | 0 dB |
| (NR2) | | thyroid | Immune | | POSTTX | 55 | 75 | 95 | 95 | 90 | 95 | |
| | | | complex + | | | | | | | | | |
| 61HF^{B} | YES | NC | NA | NEG | PRETX | 65 | 60 | 40 | 35 | 45 | 70 | 0 dB |
| (NR3) | | | | | POSTTX | 70 | 60 | 50 | 35 | 45 | 60 | |
| 57 WM^{B} | YES | Brother | ANA1:2560, | NEG | PRETX | 55 | 60 | 75 | 65 | 80 | 85 | 0 dB |
| (NR4) | | W/ Crohns | P0 pos | | POSTTX | 60 | 70 | 75 | 70 | 75 | 70 | |
| 45WM | YES | NC | RF borderline + | NEG | PRETX | 60 | 60 | 65 | 65 | 70 | 80 | 1 dB |
| (NR 5) | | | | | POSTTX | 60 | 60 | 70 | 60 | 60 | 85 | (NS) |
| 62 WF | YES | NC | Weak + 68 kD | NEG | PRETX | 10 | 10 | 10 | 10 | 40 | 35 | 0dB |
| (NR6) | | | Borderline | | POSTTX | 15 | 15 | 10 | 10 | 55 | 70 | |
| | | | typeII collagen | | | | | | | | | |
| 60WF | YES | Pulm | P0 pos | NEG | PRETX | 60 | 60 | 60 | 55 | 75 | 110 | 4 dB |
| (NR7) | | collag vasc | | | POSTTX | 65 | 60 | 65 | 55 | 70 | 80 | (NS) |
| | | disease | | | | | | | | | | |
| 56 WF^{B} | YES | Daughter | ANA 1:640, | NEG | PRETX | 55 | 60 | 60 | 40 | 40 | 75 | 38 dB |
| (R1) | | W/ IDDM | Phospholipid | | POSTTX | 15 | 20 | 15 | 5 | 15 | 40 | |
| | | | IgG & IgM pos | | | | | | | | | |
| 54 WF^{B} | YES | Hashimotos | ANA 1:160, | NEG | PRETX | 105 | 115 | 100 | 100 | 100 | 105+ | 33 dB |
| (R2) | | Thyroiditis | 68 kD pos | | POSTTX | 40 | 60 | 70 | 80 | 75 | 105+ | |
| 39WM^{B} | YES | NC | NA | NEG | PRETX | 40 | 45 | 35 | 15 | 30 | 30 | 13 dB |
| (R3) | | | | | POSTTX | 25 | 15 | 5 | 5 | 40 | 25 | |

Clinical history and audiometric data of patients treated with prednisone.
“Prior tx?”: although all had prior rapid declines in hearing requiring prednisone therapy, none were treated in a 3 month period prior to enrollment. Several patients had occasional vestibular symptoms; however, these symptoms did not coincide with periods of hearing fluctuation.
Serology was performed at Immco Diagnostics.
NA = not available: serologic testing at Immco was not covered by the patient's insurance carrier.
NS = not significant: average post-treatment hearing changes of less than 10dB are not felt to represent significant changes. All declines in hearing are reported as 0 dB.
NR = clinical non-responder;
R = clinical responder.
The superscript “B” refers to inclusion in FIG 5B.

### Discussion

IL-1RII is a member of the IL-1 receptor family and is a decoy receptor that functions as a molecular trap for IL-113 (reviewed in Manotvani et al., 2001). Induction of IL-1RII and interleukin-1 receptor type antagonist (IL-1RA) have been proposed as mechanisms that maintain site-specific immunoprivilege by preventing IL-1β mediated inflammation. In the aqueous humor of the eye, IL-IRA is upregulated (Kennedy et al., 1995). In a rat model, intracerebral injection of IL-β induced a rapid preferential transcription of IL-1RII over IL-1R1 (Docagne et al., 2005). The observed up-regulation of IL-1RII by PBMC exposed to cochlear fluid suggests the inner ear is an immunoprivileged site as well. PBMC that traffic to the inner ear normally express IL1-RII and prevent inflammation. Patients with AIED are unable to mount a similar response, and unopposed IL-1βinflammation ensues. Unlike controls, patients with AIED have been reported to have autoreactive T-cells to inner ear homogenates (Hughes et al., 1986). It may be that repetitive exposure to inner ear antigens changes the phenotype of the responding T-cells and ultimately renders them refractory to IL-1RII upregulation. In support of such a hypothesis, poor steroid response correlates with increased numbers of CD4+CD45RO+ memory cells Garcia-Berrocal et al., 1997). Activation of the innate immune response may prime adaptive immunity. Controls primarily have an innate response, whereas those with AIED have been previously primed and respond with a heightened adaptive response (Hashimoto et al., 2005).

In other systems, levels of the IL-1RII are up-regulated after systemic steroid therapy (Muller et al., 2002). Steroids have been the mainstay for recovery and stabilization of hearing in patients with autoimmune or other sudden hearing loss (Broughton et al., 2004). Experimentally, methotrexate stimulates IL-1RA release and inhibits IL-1β synthesis (Seitz et al., 1998). However, a recent large trial demonstrated methotrexate could not maintain hearing compared to placebo (Harris et al., 2003). This poor clinical response may be a result of IL-1RA inciting death of spiral ganglion neurons in the inner ear (Komeda et al., 1999). IL-1Rn has been used successfully to reduce inflammation in a murine model of collagen-induced arthritis (Bessis et al., 2000).

The present results demonstrate the involvement of the Interleukin-1 receptor type 2 in corticosteroid-sensitive AIED and the amelioration and/or progression of immune mediated hearing loss. Interestingly, the clear absent mRNA expression of the membrane bound form in the AIED patients despite expression of the shorter soluble form suggests alternative splicing in these patients with a likely introduction of a stop codon as described by Liu et al (1996). Induction of expression of the membrane bound decoy receptor in response to glucocorticoids during periods of inflammation and rapid hearing decline correlates with clinical restoration of hearing to baseline thresholds suggests a change in transcriptional control. The robust expression of mIL-1RII in control cochlear implant subjects and those that experienced corticosteroid-induced hearing recovery suggest that the inner ear likely is a functionally immunoprivileged site. Moreover, expression of IL-1M in response to perilymph in control subjects suggests that, under normal conditions, the inner ear is tolerant of local inflammation.

The absence of basal expression of mIL-1RII in both patients undergoing steroid therapy for sudden hearing declines and cochlear implantation suggests that IL-1RII is a critical regulatory protein in hearing homeostasis. Patients who are corticosteroid-responsive during sudden hearing declines, likely represent early AIED, whereas those undergoing cochlear implantation, who have end stage disease, are no longer corticosteroid-responsive. The observations described here indicate that mIL-1RII expression may be an important mechanism involved in corticosteroid responsiveness in AIED, which is a poorly understood disorder of the inner ear.

The ability to predict corticosteroid responsiveness by differential expression of IL-1RII as set forth herein provides 1) a diagnostic test for AIED, 2) a predictive test for which patients with AIED or other autoimmune diseases or disorders will respond to glucocorticoids, thereby avoiding undue risk associated with corticosteroid treatment in non-responders, 3) the basis for novel therapies through restoration of IL1R2 expression, and 4) a rationale to develop novel therapies to treat other corticosteroid-sensitive autoimmune diseases.

### REFERENCES

Bessis N, Guery L, Mantovani A, et al. The type II decoy receptor of IL-1 inhibits murine collagen-induced arthritis. Eur J Immunol 2000; 30: 867-75.
Broughton S S, Meyerhoff W E, Cohen S B. Immune mediated inner ear disease: 10-year experience. Semin Arthritis Rheum. 2004; 34: 544-8.
Cao M Y, Dupriez V J, Rider M H, et al. Myelin protein Po as a potential autoantigen in autoimmune inner ear disease. FASEB 1996; 10: 1635-40.
Docagne F, Campbell S J, Bristow A F, et al. Differential regulation of type I and type II interleukin-1 receptors in focal brain inflammation. Eur J Neurosci 2005; 21: 1205-14.
Garcia-Berrocal J R, Vargas J A, Ramirez-Camacho R A, et al. Deficiency of naïve T cells in patients with sudden deafness. Arch Otolaryngol Head Neck Surg 1997; 123: 712-7.
Gloddek B, Arnold W. Clinical and experimental studies of autoimmune inner ear disease. Acta Otolaryngol Suppl. 2002; 548: 10-14.
Harris J P, Weissman M H, Derebery J M et al. Treatment of corticosteroid-responsive autoimmune inner ear disease with methotrexate: a randomized controlled trial. JAMA 2003; 290: 1875-83.
Hashimoto S, Billings P, Harris J P, et al. Innate Immunity Contributes to Cochlear Adaptive Immune Responses. Audiology & Neurotology. 2005; 10: 35-43.
Hughes G B, Barna B P, Kinney S E, et al. Predictive value of laboratory tests in “autoimmune” inner ear disease: preliminary report. Laryngoscope 1986; 96:502-5.
Iwai H, Tomoda K, Sugiura K, et al. T cells infiltrating from the systemic circulation proliferate in the endolymphatic sac. Ann Otol Rhinol Laryngol. 1999; 108: 1146-50.
Kennedy M C, Rosenbaum J T, Brown J. Novel production of interleukin-1 receptor antagonist peptides in normal human cornea. J Clin Invest 1995; 95: 82-88.
Komeda M, Roessler B J, Raphael Y. The influence of interleukin-1 receptor antagonist transgene on spiral ganglion neurons. Hear Res. 1999; 131: 1-10.
Lin D W, Trune D R. Breakdown of stria vascularis blood-labyrinth barrier in C3H/lpr autoimmune disease mice. Otolaryngol Head Neck Surg. 1997; 117: 530-4.
Liu C, Hart R P, Liu X J, Clevenger W, Maki R A, DeSouza E B. Cloning and characterization of an alternatively processed human type II interleukin-1 receptor mRNA. J Biol Chem 1996; 271(34): 20965-72.
Manotvani A, Locati M, Vecchi A, et al. Decoy receptors: a strategy to regulate inflammatory cytokines and chemokines. Trends in Immunol 2001; 22: 328
Mantovani A, Muzio M, Ghezzi P, Colotta C, Introna M. Regulation of Inhibitory Pathways of the Interleukin-1 System. Ann N Y Acad Sci 1998; 840: 338-51.
Mogi, G, Kawauchi H, Suzuki M, et al. Inner ear immunology. Am J. Otolaryngol. 1985; 6: 142-7.
Moscicki R A, San Martin J E, Quintero C H, et al. Serum antibody to inner ear proteins in patients with progressive hearing loss. Correlation with disease activity and response to corticosteroid treatment. JAMA 1994; 272: 611-6.
Muller B, Peri G, Doni A, et al. High circulating levels of the IL-1 type II decoy receptor in critically ill patients with sepsis: association of high decoy receptor levels with glucocorticoid administration. J Leukoc Biol 2002; 72: 643-9.
Neumann D, Kollewe C, Martin M U, Boraschi D. The membrane bound for of the type II IL-1 receptor accounts for inhibitory function. J. Immunol. 2000; 165: 3350-7.
Niparko J K, Wang N Y, Rauch S D, Russell G B, Espeland M A, Pierce J J, Bowditch S, Masuda A, Gulya A J, Gantz B J, Hughes G B, Brookhouser P E, Hannley M T, Telian S A, Harris J P, AIED study group. Serial audiometry in a clinical trial of AIED treatment. Otol Neurotol 2005; 26: 908-17.
Quaranta N, Bartoli R, Giagnotti F, et al. Cochlear implants in systemic autoimmune vasculitis syndromes. Acta Otolaryngol Supp 2002; 549: 44-8.
Ruckenstein M J. Autoimmune inner ear disease. Curr Opin Otolaryngol Head Neck Surg 2004; 12: 426-30.
Ryan A F, Harris J P, Keithley E M. Immune-mediated hearing loss: basic mechanisms and options for therapy. Acta Otolaryngol. Supp. 2002; 548: 38-43.
Satoh H, Firestein G S, Billings P B, et al. Tumor necrosis factor alpha, an initiator, and etanercept, an inhibitor of cochlear inflammation. Laryngoscope 2002; 112: 1627-34.
Seitz M, Zwicker M, Loetscher P. Effects of methotrexate on differentiation of monocytes and production of cytokine inhibitors by monocytes. Arthritis Rheum 1998; 41: 2032-2038.
Strausberg R L, Feingold E A, Grouse L H, Derge J G, Klausner R D, et al. Mammalian Gene Collection Program Team. Generation and initial analysis of more than 15,000 full-length human and mouse cDNA sequences. Proc. Natl. Acad. Sci. U.S.A. 99 (26), 16899-16903 (2002).
Trune D R, Wolbig R J, Kempton J B, et al. Steroid treatment improves cochlear function in the MRL.MpJ Fas (lpr) autoimmune mouse. Hear Res. 1999; 137: 160-6.
Wuorimaa T, Kayhty H, Eskola J, et al. Activation of cell mediated immunity following immunization with pneumococcal conjugate or polysaccharide vaccine. Scand J. Immunol. 2001; 53: 422-428.
Yeo S W, Gottschlich S, Harris J P, et al. Antigen diffusion from the perilymphatic space of the cochlea. Laryngoscope 1995; 105: 623-8.
Yeom K, Gray J, Nair T S, Arts H A, Telian S A, Disher M J, El-Kashlan H, Sataloff R T, Fisher S G, Carey T E. Antibodies to HSP-70 in normal donors and autoimmune hearing loss patients. Laryngoscope 2003; 113: 1770-6.

1. A method of determining whether or not a patient will respond to steroid treatment, the method comprising
(a) treating mononuclear immune cells from the patient with a glucocorticoid;
(b) measuring membrane-bound interleukin-1 receptor type II (mIL-1RII) levels in a sample of the patient's mononuclear immune cells before and after treatment with the glucocorticoid; and
(c) comparing the mIL-1RII levels in the cells before and after glucocorticoid treatment,
wherein an increase in mIL-1RII levels after glucocorticoid treatment above a response observed from mononuclear immune cells of non-responding subjects indicates that the patient will respond to steroid treatment, or
wherein a change in mIL-1RII levels after glucocorticoid treatment that is not greater than a response observed from mononuclear immune cells of non-responding subjects indicates that the patient will not respond to steroid treatment.

2. The method of claim 1, wherein the change in mIL-1RII in the sample from the patient is compared to the response from mononuclear immune cells of subjects who are known to respond to steroid treatment.

3. The method of claim 1, wherein the change in mIL-1RII in the sample from the patient is compared to the change in mIL-1RII from mononuclear immune cells of subjects who are known to not respond to steroid treatment.

4. The method of claim 1, wherein the amount of increase in mIL-1RII mRNA levels is determined by
comparing the response from the patient with the response from mononuclear immune cells of subjects who are known to respond to steroid treatment and
comparing the response from the patient with the response from mononuclear immune cells of subjects who are known to not respond to steroid treatment.

5. The method of claim 1, wherein an increase in mIL-1RII levels in the cells after glucocorticoid treatment of at least 10,000-fold indicates that the patient will respond to steroid treatment.

6. The method of claim 1, wherein an increase in mIL-1RII levels in the cells after glucocorticoid treatment of at least 100,000-fold indicates that the patient will respond to steroid treatment.

7. The method of claim 1, wherein an increase in mIL-1RII levels in the cells after glucocorticoid treatment of at least 1,000,000-fold indicates that the patient will respond to steroid treatment.

8. The method of claim 1, wherein an increase in mIL-1RII levels in the cells after glucocorticoid treatment of 100-fold or less indicates that the patient not will respond to steroid treatment.

9. The method of claim 1, wherein an increase in mIL-1RII levels in the cells after glucocorticoid treatment of 500-fold or less, or 1,000-fold or less, indicates that the patient not will respond to steroid treatment.

10. A method of determining whether or not a patient will respond to steroid treatment, the method comprising
measuring for basal membrane-bound interleukin-1 receptor type II (mIL-1RII) in a sample of the patient's mononuclear immune cells,
wherein a failure to detect mIL-1RII indicates the patient will respond to steroid treatment, or
wherein detection of a level of mIL-1RII below the level from mononuclear immune cells of subjects who are known to not respond to steroid treatment indicates the patient will respond to steroid treatment.

11. The method of claim 1, wherein the IL-1RII levels are determined by determining IL-1RII mRNA levels in the cells.

12. The method of claim 10, wherein 40 cycles or more of quantitative real-time polymerase chain reaction (Q-RT-PCR) are required to detect mIL-1RII, or fail to detect mIL-1RII, indicating that the patient will respond to steroid treatment.

13. The method of claim 1, wherein the IL-1RII levels are determined by determining IL-1RII protein levels in the cells.

14. The method of claim 1, wherein the mononuclear immune cells are peripheral blood mononuclear cells (PBMCs).

15. The method of claim 1, wherein the glucocorticoid is dexamethasone.

16. The method of claim 1, wherein the IL-1RII levels are determined by determining IL-1RII mRNA levels in the cells, the mononuclear immune cells are PBMCs and the glucocorticoid is dexamethasone.

17. The method of claim 1, wherein the patient has an autoimmune disease or disorder.

18. The method of claim 17, wherein the autoimmune disease or disorder is an inflammatory bowel disease, rheumatoid arthritis, systemic lupus erythematosus, asthma, autoimmune inner ear disease, or transplant rejection.

19. The method of claim 18, wherein the inflammatory bowel disease is ulcerative colitis.

20. The method of claim 18, wherein the transplant rejection is renal transplant rejection.

21. A method for treating a subject having autoimmune inner ear disease, sudden sensorineural hearing loss, Ménière's disease, or acoustic trauma comprising administering to the subject an effective amount of an IL-1 inhibitor or antagonist.
